(19) United States
(12) Patent Application Publication
(10) Pub. No.: US 2006/0178370 A1
(43) Pub. Date: Aug. 10, 2006

(54) Ketopiperazine derivatives as bradykinin antagonists

(76) Inventors: Mark Bock, Hatfield, PA (US); Bruce Dorsey, Ambler, PA (US); Dai-Shi Su, Dresher, PA (US); Wei Han, West Chester, PA (US); Michael Wood, Harleysville, MA (US)

Correspondence Address: MERCK AND CO., INC, P O BOX 2000, RAHWAY, NJ, 07065-0907, US

(21) Appl. No.: 10/548,155

(22) PCT Filed: Mar. 15, 2004
(86) PCT No.: PCT/US04/07942

§ 371 Date: Sep. 6, 2005

Related U.S. Application Data
(60) Provisional application No. 60/455,681, filed on Mar. 18, 2003.

(51) Int. Cl. A61K 31/496; A61K 31/495; C07D 403/02; C07D 241/06; C07D 241/08

(52) U.S. Cl. 514/252.13; 514/255.02; 544/359; 544/384

(57) ABSTRACT

Ketopiperazine derivatives are bradykinin B1 antagonists or inverse agonists useful in the treatment or prevention of symptoms such as pain and inflammation associated with the bradykinin B1 pathway.

### BACKGROUND OF THE INVENTION

This invention is directed to ketopiperazine compounds. In particular, this invention is directed to ketopiperazine compounds that are bradykinin antagonists or inverse agonists.

Bradykinin (“BK”) is a kinin which plays an important role in the pathophysiological processes accompanying acute and chronic pain and inflammation. Bradykinin (BK), like other kinins, is an autacoid peptide produced by the catalytic action of kallikrein enzymes on plasma and tissue precursors termed kinihogens. The biological actions of BK are mediated by at least two major G-protein-coupled BK receptors termed B1 and B2. It is generally believed that B2 receptors, but not B1 receptors, are expressed in normal tissues and that inflammation, tissue damage or bacterial infection can rapidly induce B1 receptor expression. This makes the B1 receptor a particularly attractive drug target. The putative role of kinins, and specifically BK, in the management of pain and inflammation has provided the impetus for developing potent and selective BK antagonists. In recent years, this effort has been heightened with the expectation that useful therapeutic agents with analgesic and anti-inflammatory properties would provide relief from maladies mediated through a BK receptor pathway (see e.g., M. G. Bock and J. Longmore, Current Opinion in Chem. Biol., 4:401-406(2000)). Accordingly, there is a need for novel compounds that are effective in blocking or reversing activation of bradykinin receptors. Such compounds would be useful in the management of pain and inflammation, as well as in the treatment or prevention of diseases and disorders mediated by bradykinin; further, such compounds are also useful as research tools (in vivo and in vitro).

### SUMMARY OF THE INVENTION

The present invention provides novel ketopiperazine derivatives which are bradykinin antagonists or inverse agonists, pharmaceutical compositions containing such compounds, and methods of using them as therapeutic agents.

### DETAILED DESCRIPTION OF THE INVENTION

The present invention describes compounds of formula I and pharmaceutically acceptable salts thereof: 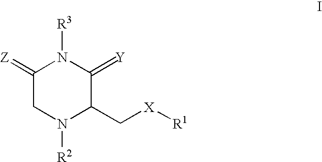
wherein
X is selected from (1) —(CH₂)ₚC(O)NR^{b}—, (2) —(CH₂)ₚNR^{b}C(O)—, (3) —(CH₂)ₚNR^{b}—, (4) —(CH₂)ₚO—, (5) —C(O)—, (6) —(CH₂)ₚC(O)O—, (7) —(CH₂)ₚS(O)ₘ—, (8) —(CH₂)ₚS—, (9) HC═CH, and (10) —(CH₂)ₚ—;
one of Y or Z is oxygen and the other represents (H,H);
R¹ is selected from (1) CH₂(CH₂)ₙOR^{a}, (2) CH₂(CH₂)ₙNR^{b}R^{c}, (3) CH₂(CH₂)ₙCN, (4) C(O)OR^{a}, (5) (CH₂)ₙC(O)OR^{a}, (6) (CH₂)ₙC(O)NR^{b}R^{c}, (7) C₁₋₆ alkyl-Q wherein Q is (a) heterocyclyl optionally substituted with 1 to 3 groups independently selected from halogen, C₁₋₄ alkyl, C₁₋₄ halogen substituted alkyl, nitro, cyano, OR^{a}, and NR^{b}R^{c}; or (b) aryl optionally substituted with 1 to 3 groups independently selected from C₁₋₄ alkyl (optionally substituted with OR^{a}, NR^{b}R^{c}, NR^{b}C(O)R^{a}, or heterocyclyl), halogen, cyano, C(O)R^{a}, C(O)OR^{a}, OR^{a}, NR^{b}R^{c}, NR^{b}C(O)R^{a}, C(O)NR^{b}R^{c}, SR^{a}, S(O)ₘR^{a′}, aryl and heterocyclyl wherein said aryl and heterocyclyl are optionally substituted with 1 to 3 groups independently selected from C₁₋₄alkyl, C(O)OR^{a}, halogen, nitro, cyano, OR^{a}, and NR^{b}R^{c}; and (8) aryl optionally substituted with 1 to 3 groups independently selected from halogen, nitro, cyano, C(O)OR^{a}, OR^{a}, NR^{b}R^{c}, NR^{b}C(O)R^{a}, C(O)NR^{b}R^{c}, SR^{a}, S(O)ₘR^{a′}, (CH₂)ₙOR^{a}, (CH₂)ₙNR^{b}R^{c}, (CH₂)ₙNR^{b}C(O)OR^{a}, O(CH₂)ₙNR^{b}R^{c}, O(CH₂)ₙNR^{b}C(O)OR^{a}, C₁₋₄ alkyl, aryl and heterocyclyl wherein said aryl and heterocyclyl are optionally substituted with 1 to 3 groups independently selected from C₁₋₄alkyl, C(O)OR^{a}, halogen, nitro, cyano, OR^{a}, and NR^{b}R^{c}; or
R¹, R^{b} and the nitrogen atom to which they are both attached together form a 4-7-membered ring optionally containing a heteroatom selected from O, S and N—R^{d};
R² is selected from (1) (CH₂)ₙC(O)OR^{a}, (2) (CH₂)ₙC(O)NR^{b}R^{c}, (3) S(O)ₘR^{a′}, (4) C(O)R^{a}, (5)° C(O)OR^{a}, (6) C₁₋₄ alkyl, (7) C₁₋₄ halogen substituted alkyl, (8) C₁₋₄ alkyl substituted with cyano, (9) C₁₋₄ alkyl substituted with an aryl optionally substituted with 1 to 3 groups independently selected from halogen, cyano, OR^{a}, NR^{b}R_{c}, C(O)NR^{b}R^{c}, and phenyl optionally substituted with 1 to 3 groups independently selected from C(O)OR^{a}, halogen, nitro, cyano, OR^{a}, and NR^{b}R^{c};
R³ is selected from (1) hydrogen, (2) C₁₋₄ alkyl, (3) C₁₋₄ halogen substituted alkyl, (4) C₁₋₄ alkyl-aryl, (5) S(O)ₘR^{a′}, (6) C₃₋₇ cycloalkyl, (7) (CH₂)ₚC(O)R^{a}, (8) (CH₂)ₚC(O)OR^{a}, and (9) (CH₂)ₚC(O)NR^{b}R^{c};
R^{a} is selected from (1) hydrogen, (2) C₁₋₄ alkyl, (3) C₁₋₄ halogen substituted alkyl, (4) C₃₋₇ cycloalkyl, (5) aryl optionally substituted with 1 to 3 groups independently selected from C₁₋₄ alkyl, halogen, nitro, cyano, OR^{d}, O—C₁₋₄ halogen substituted alkyl, and NR^{b}R^{c}, and (6) heteroaryl optionally substituted with 1 to 3 groups independently selected from C₁₋₄ alkyl, halogen, nitro, cyano, OR^{d}, O—C₁₋₄ halogen substituted alkyl, and NR^{b}R^{c};
R^{a′} is selected from (1) C₁₋₄ alkyl, (2) C₁₋₄ halogen substituted alkyl, (3) C₃₋₇ cycloalkyl, (4) aryl optionally substituted with 1 to 3 groups independently selected from C₁₋₄ alkyl, halogen, nitro, cyano, OR^{d}, O—C₁₋₄ halogen substituted alkyl, and NR^{b}R^{c}, and (5) heteroaryl optionally substituted with 1 to 3 groups independently selected from C₁₋₄ alkyl, halogen, nitro, cyano, OR^{d}, O—C₁₋₄ halogen substituted alkyl, and NR^{b}R^{c};
R^{b} and R^{c} are independently selected from (1) hydrogen, (2) C₁₋₄ alkyl, (3) C₁₋₁₄ halogen substituted alkyl, (4) C₃₋₇ cycloalkyl, and (5) aryl optionally substituted with 1 to 3 groups independently selected from C₁₋₄ alkyl, halogen, nitro, cyano, OR^{d}, O—C₁₋₄ halogen substituted alkyl, —C(O)OR^{d}, C(O)NR^{d}R_{d}, and NR^{d}R^{d}; or
R^{b}, R^{c} and the nitrogen atom to which they are attached together form a 4-7-membered ring optionally containing a heteroatom selected from O, S and N—R^{d},
R^{d} is hydrogen or C₁₋₄ alkyl;
n is an integer from 1 to 6;
m is 1 or 2;
p is 0 to 6;
with the proviso that when X is —CH₂)ₚ—, R¹ is (a) C₁₋₆ alkyl-heterocycle optionally substituted with 1 to 3 groups independently selected from halogen, C₁₋₄ alkyl, C₁₋₄ halogen substituted alkyl, nitro, cyano, OR^{a}, and NR^{b}R^{c}, or (b) aryl substituted with a heterocycle which is optionally substituted with 1 to 3 groups independently selected from C₁₋₄alkyl, C(O)OR^{a}, halogen, nitro, cyano, OR^{a}, and NR^{b}R^{c}.

In one subset of formula I are compounds wherein Y is O and Z is (H,H).

In a second subset of formula I are compounds wherein R³ is H.

In a third subset of formula I are compounds wherein X is —(CH₂)ₚC(O)NR^{b}— or —(CH₂)ₚS(O)ₘ. In one embodiment thereof are compounds wherein X is —CONH—; in a second embodiment thereof X is SO₂.

In a fourth subset of formula I are compounds wherein R² is S(O)ₘR^{a′} or C(O)R^{a}. In one embodiment thereof R² is SO₂R^{a′}. In a further embodiment thereof R² is SO₂-aryl optionally substituted with 1 to 3 groups independently selected from C₁₋₄ alkyl, halogen, nitro, cyano, OR^{d}, O—C₁₋₄ halogen substituted alkyl, and NR^{b}R^{c}. In yet a further embodiment thereof aryl is 5-, 6-, 7-, 8-(1,2,3,4-tetrahydroquinolinyl), naphthyl or phenyl substituted with halogen. In yet another embodiment thereof R² is SO₂-(2-naphthyl). In another embodiment thereof R² is SO₂-(1,2,3,4-tetrahydroquinolin-8-yl).

In a fifth subset of formula I are compounds wherein R¹ is selected from (1) CH₂(CH₂)ₙO-aryl where aryl is optionally substituted with 1 to 3 groups independently selected from C₁₋₄ alkyl, halogen, nitro, cyano, OR^{d}, O—C₁₋₄ halogen substituted alkyl, and NR^{b}R^{c}, (2) CH₂(CH₂)ₙO-heteroaryl where heteroaryl is optionally substituted with 1 to 3 groups independently selected from C₁₋₄ alkyl, halogen, nitro, cyano, OR^{d}, O—C₁₋₄ halogen substituted alkyl, and NR^{b}R^{c}, (3) CH₂(CH₂)ₙNR^{b}R^{c}, (4) C₁₋₆ alkyl-Q wherein Q is (a) heterocyclyl optionally substituted with 1 to 3 groups independently selected from halogen, C₁₋₄ alkyl, C₁₋₄ halogen substituted alkyl, nitro, cyano, OR^{a}, and NR^{b}R^{c}; or (b) aryl optionally substituted with 1 to 3 groups independently selected from CIA alkyl (optionally substituted with OR^{a}, NR^{b}R^{c}, NR^{b}C(O)R^{a}, or heterocyclyl), halogen, cyano, C(O)R^{a}, C(O)OR^{a}, OR^{a}, NR^{b}R^{c}, NR^{b}C(O)R^{a}, C(O)NR^{b}R^{c}, SR^{a}, S(O)ₘR^{a′}, aryl and heterocyclyl wherein said aryl and heterocyclyl are optionally substituted with 1 to 3 groups independently selected from C₁₋₄alkyl, C(O)OR^{a}, halogen, nitro, cyano, OR^{a}, and NR^{b}R^{c}, and (5) aryl optionally substituted with 1 to 3 groups independently selected from halogen, nitro, cyano, C(O)OR^{a}, OR^{a}, NR^{b}R^{c}, NR^{b}C(O)R^{a}, C(O)NR^{b}R^{c}, SR^{a}, S(O)ₘR^{a′}, (CH₂)ₙOR^{a}, (CH₂)ₙNR^{b}R^{c}, (CH₂)ₙNR^{b}C(O)OR^{a}, O(CH₂)ₙNR^{b}R^{c}, O(CH₂)ₙNR^{b}C(O)OR^{a}, C₁₋₄ alkyl, aryl and heterocyclyl wherein said aryl and heterocyclyl are optionally substituted with 1 to 3 groups independently selected from C₁₋₄alkyl, C(O)OR^{a}, halogen, nitro, cyano, OR^{a}, and NR^{b}R^{c}.

In one embodiment of the fifth subset, R¹ is CH₂(CH₂)ₙO-heteroaryl where heteroaryl is optionally substituted as defined above, and n is 1 to 5. In one subgroup R¹ is (CH₂)₂₋₆-O-pyridyl.

In a second embodiment of the fifth subset R¹ is C₁₋₆ alkyl-heterocyclyl wherein said heterocyclyl is optionally substituted with 1 to 3 groups independently selected from halogen, C₁₋₄ alkyl, C₁₋₄ halogen substituted alkyl, nitro, cyano, OR^{a}, and NR^{b}R^{c}. In one subgroup R¹ is (CH₂)₂₋₆-heterocyclyl where said heterocyclyl is pyridyl optionally substituted with 1 to 3 groups independently selected from halogen, C₁₋₄ alkyl, C₁₋₄ halogen substituted alkyl, nitro, cyano, OR^{a}, and NR^{b}R^{c}. In a further subgroup R¹ is (CH₂)₂₋₆-heterocycle where the heterocycle is naphthyridine or tetrahydronaphthyridine such as 1,8-naphthyridin-2-yl or 5,6,7,8-tetrahydro-1,8-naphthyridin-2-yl.

In a third embodiment of the fifth subset R¹ is C₁₋₆ alkyl-aryl wherein the aryl group is optionally substituted with 1 to 3 groups independently selected from C₁₋₄ alkyl (optionally substituted with OR^{a}, NR^{b}R^{c}, NR^{b}C(O)R^{a}, or heterocyclyl), halogen, cyano, C(O)H, OR^{a}, NR^{b}R_{c}, NR^{b}C(O)R^{a}, C(O)NR^{b}R_{c}, SR^{a}, S(O)ₘR^{a′}, aryl and heterocyclyl wherein said aryl and heterocyclyl are optionally substituted with 1 to 3 groups independently selected from C₁₋₄alkyl, C(O)OR^{a}, halogen, nitro, cyano, OR^{a}, and NR^{b}R^{c}. In one subgroup R¹ is (CH₂)₂₋₆-phenyl wherein phenyl is substituted with N—R^{d}-imidazoline, N—R^{d}-imidazole or N—R^{d}-triazole. In another subgroup R¹ is (CH₂)₂₋₆-phenyl wherein phenyl is substituted with cyano or C₁₋₄ alkyl substituted with a group selected from NR^{b}R^{c} and NR^{b}C(O)R^{a}.

In a fourth embodiment of the fifth subset R¹ is aryl optionally substituted with 1 to 3 groups independently selected from halogen, nitro, cyano, C(O)OR^{a}, OR^{a}, NR^{b}R^{c}, SR^{a}, S(O)ₘR^{a′}, (CH₂)ₙOR^{a}, (CH₂)ₙNR^{b}R^{c}, (CH₂)ₙNR^{b}C(O)OR^{a}, O(CH₂)ₙNR^{b}R^{c}, O(CH₂)ₙNR^{b}C(O)OR^{a}, C₁₋₄ alkyl, aryl and heterocyclyl wherein said aryl and heterocyclyl are optionally substituted with 1 to 3 groups independently selected from C₁₋₄alkyl, C(O)OR^{a}, halogen, nitro, cyano, OR^{a}, and NR^{b}R^{c}. In one subgroup R¹ is phenyl substituted with 4-(1-piperidyl)-1-piperidyl, O(CH₂)ₙNR^{b}R^{c} or (CH₂)ₙNR^{b}R^{c}. In another subgroup R¹ is 4-[4-(1-piperidyl)-1-piperidyl]phenyl.

In a sixth subset of formula I are compounds of formula Ia: 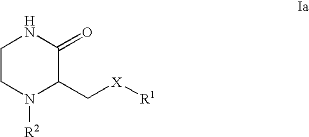
wherein
X is —C(O)NH— or SO₂;
R¹ is selected from (1) CH₂(CH₂)ₙO-aryl where aryl is optionally substituted with 1 to 3 groups independently selected from C₁₋₄ alkyl, halogen, nitro, cyano, OR^{d}, O—C₁₋₄ halogen substituted alkyl, and NR^{b}R^{c}, (2) CH₂(CH₂)ₙO-heteroaryl where heteroaryl is optionally substituted with 1 to 3 groups independently selected from C₁₋₄ alkyl, halogen, nitro, cyano, OR^{d}, O—C₁₋₄ halogen substituted alkyl, and NR^{b}R^{c}, (3) CH₂(CH₂)ₙNR^{b}R^{c}, (4) C₁₋₆ alkyl-Q wherein Q is (a) heterocyclyl optionally substituted with 1 to 3 groups independently selected from halogen, C₁₋₄ alkyl, C₁₋₄ halogen substituted alkyl, nitro, cyano, OR^{a}, and NR^{b}R^{c}; or (b) aryl optionally substituted with 1 to 3 groups independently selected from C₁₋₄ alkyl (optionally substituted with OR^{a}, NR^{b}R^{c}, NR^{b}C(O)R^{a}, or heterocyclyl), halogen, cyano, C(O)R^{a}, C(O)OR^{a}, OR^{a}, NR^{b}R^{c}, NR^{b}C(O)R^{a}, C(O)NR^{b}R^{c}, SR^{a}, S(O)ₘR^{a′}, aryl and heterocyclyl wherein said aryl and heterocyclyl are optionally substituted with 1 to 3 groups independently selected from C₁₋₄alkyl, C(O)OR^{a}, halogen, nitro, cyano, OR^{a}, and NR^{b}R^{c}, and (5) aryl optionally substituted with 1 to 3 groups independently selected from halogen, nitro, cyano, C(O)OR^{a}, OR^{a}, NR^{b}R^{c}, NR^{b}C(O)R^{a}, C(O)NR^{b}R^{c}, SR^{a}, S(O)ₘR^{a′}, (CH₂)ₙOR^{a}, (CH₂)ₙNR^{b}R^{c}, (CH₂)ₙNR^{b}C(O)OR^{a}, O(CH₂)ₙNR^{b}R^{c}, O(CH₂)ₙNR^{b}C(O)OR^{a}, C₁₋₄ alkyl, aryl and heterocyclyl wherein said aryl and heterocyclyl are optionally substituted with 1 to 3 groups independently selected from C₁₋₄alkyl, C(O)OR^{a}, halogen, nitro, cyano, OR^{a}, and NR^{b}R^{c}; and
R² is (1) SO₂-aryl optionally substituted with 1 to 3 groups independently selected from C₁₋₄ alkyl, halogen, nitro, cyano, OR^{d}, O—C₁₋₄ halogen substituted alkyl, and NR^{b}R^{c}, or (2)C(O)-aryl optionally substituted with 1 to 3 groups independently selected from C₁₋₄ alkyl, halogen, nitro, cyano, OR^{d}, O—C₁₋₄ halogen substituted alkyl, and NR^{b}R^{c}.

In one embodiment of formula Ia R² is SO₂-aryl optionally substituted with 1 to 3 groups independently selected from C₁₋₄ alkyl, halogen, nitro, cyano, OR^{d}, O—C₁₋₄ halogen substituted alkyl, and NR^{b}R^{c}. In a further embodiment R² is SO₂-aryl wherein aryl is 5-, 6-, 7- or 8-(1,2,3,4-tetrahydroquinolinyl), naphthyl or phenyl substituted with halogen. In another embodiment R² is SO₂-chlorophenyl, SO₂-(2-naphthyl) or SO₂-(1,2,3,4-tetrahydroquinolin-8-yl). In yet another embodiment R² is SO₂-(2-naphthyl). In another embodiment R² is SO₂-(1,2,3,4-tetrahydroquinolin-8-yl).

In another embodiment of formula Ia R¹ is selected from

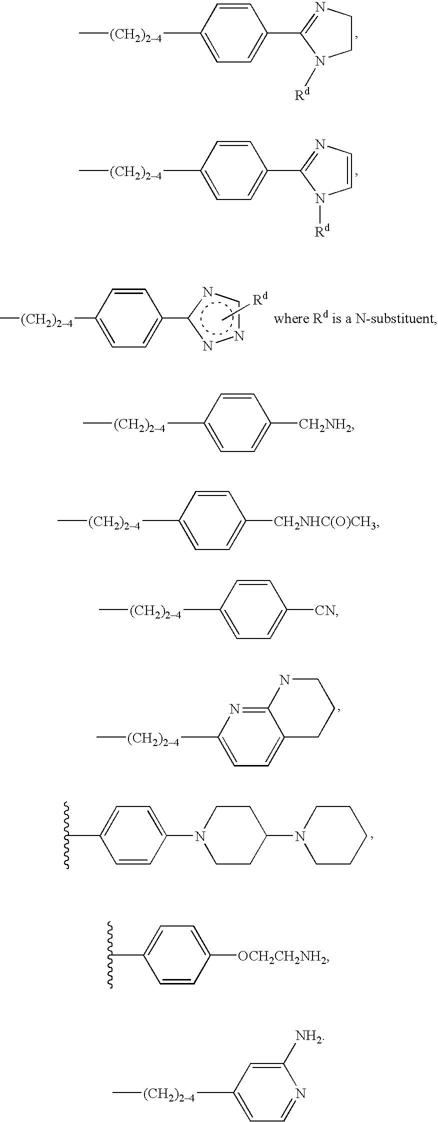

In a further embodiment of formula Ia R¹ is —(CH₂)₂₋₆—O-pyridyl.

In a seventh subset of formula I are compounds of formula Ib: 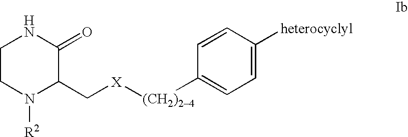
wherein R² and R^{d} are as defined under formula I; X is —CONH— or SO₂; and heterocyclyl is selected from N—R^{d}-4,5-dihydro-2-imidazolyl, N—R^{d}-2-imidazolyl, and N—R^{d}-1,2,4-triazol-3- and 5-yl. In one embodiment of formula Ib, X is —C(O)NH—. In another embodiment the heterocyclyl is N—R^{d}-4,5-dihydro-2-imidazolyl. In another embodiment R² is C(O)R^{a} or SO₂R^{a′}. In another embodiment R² is C(O)-aryl or SO₂-aryl, wherein aryl is optionally substituted with 1 to 3 groups independently selected from C₁₋₄ alkyl, halogen, nitro, cyano, OR^{d}, O—C₁₋₄ halogen substituted alkyl, and NR^{b}R^{c}. In a further embodiment R² is SO₂-aryl wherein aryl is tetrahydroquinolinyl, naphthyl or phenyl substituted with halogen. In another embodiment R² is SO₂-chlorophenyl, SO₂-(2-naphthyl) or SO₂-(1,2,3,4-tetrahydro-quinolin-8-yl). In yet another embodiment R² is SO₂-(2-naphthyl). In another embodiment R² is SO₂-(1,2,3,4-tetrahydroquinolin-8-yl).

In an eighth subset of formula I are compounds of formula Ic: 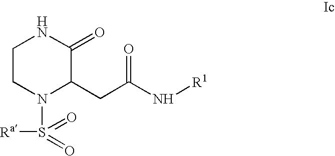
wherein R^{a′} is as defined under formula I; and R¹ is selected from

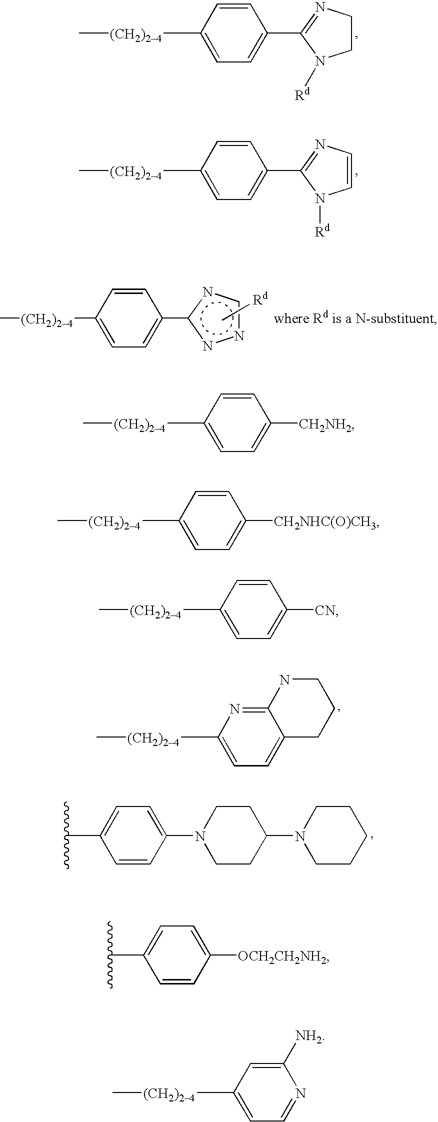

In one embodiment of formula Ic R^{a′} is 2-naphthyl. In another embodiment R^{a′} is 1,2,3,4-tetrahydroquinolin-8-yl. In yet another embodiment R^{a′} is 2-naphthyl or 1,2,3,4-tetrahydroquinolin-8-yl, and R¹ is selected from:

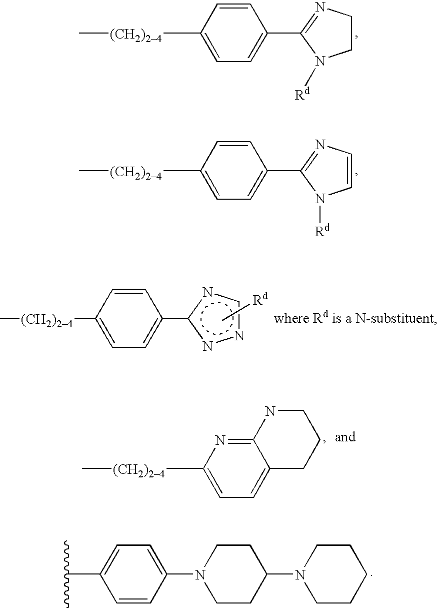

In a ninth subset of formula I are compounds of formula Id: 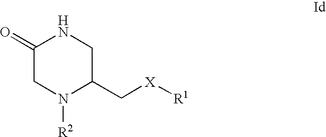
X is —C(O)NH— or SO₂;
R¹ is selected from (1) CH₂(CH₂)ₙO-aryl where aryl is optionally substituted with 1 to 3 groups independently selected from C₁₋₄ alkyl, halogen, nitro, cyano, OR^{d}, O—C₁₋₄ halogen substituted alkyl, and NR^{b}R^{c}, (2) CH₂(CH₂)ₙO-heteroaryl where heteroaryl is optionally substituted with 1 to 3 groups independently selected from C₁₋₄ alkyl, halogen, nitro, cyano, OR^{d}, O—C₁₋₄ halogen substituted alkyl, and NR^{b}R^{c}, (3) CH₂(CH₂)ₙNR^{b}R^{c}, (4) C₁₋₆ alkyl-Q wherein Q is (a) a heterocycle optionally substituted with 1 to 3 groups independently selected from halogen, C₁₋₄ alkyl, C₁₋₄ halogen substituted alkyl, nitro, cyano, OR^{a}, and NR^{b}R^{c}; and (b) an aryl group optionally substituted with 1 to 3 groups independently selected from C₁₋₄ alkyl (optionally substituted with OR^{a}, and NR^{b}R^{c}, NR^{b}C(O)R^{a}, a heterocycle selected from tetrahydro-1,8-naphthyridine, 1,8-naphthyridine, pyrazole, triazole, imidazole, piperidine, piperazine, pyridine), halogen, cyano, C(O)R^{a}, C(O)OR^{a}, OR^{a}, NR^{b}R^{c}, NR^{b}C(O)R^{a}, C(O)NR^{b}R^{c}, SR^{a}, S(O)ₘR^{a′}, morpholine, imidazoline, imidazole, triazole, piperazine, piperidine, phenyl, substituted phenyl, pyridine, substituted pyridine wherein the phenyl and pyridyl substituent(s) are 1 to 3 groups independently selected from C(O)OR^{a}, halogen, nitro, cyano, OR^{a}, and NR^{b}R^{c}, and (5) aryl optionally substituted with 1 to 3 groups independently selected from halogen, nitro, cyano, C(O)OR^{a}, OR^{a}, NR^{b}R^{c}, NR^{b}C(O)R^{a}, C(O)NR^{b}R^{c}, SR^{a}, S(O)ₘR^{a′}, (CH₂)ₙOR^{a}, (CH₂)ₙNR^{b}R^{c}, (CH₂)ₙNR^{b}C(O)OR^{a}, O(CH₂)ₙNR^{b}R^{c}, O(CH₂)ₙNR^{b}C(O)OR^{a}, morpholine, imidazoline, piperazine, piperidine optionally substituted with piperidine, imidazole, pyrazole, triazole, and C₁₋₄ alkyl; and
R² is (1) SO₂-aryl optionally substituted with 1 to 3 groups independently selected from CIA alkyl, halogen, nitro, cyano, OR^{d}, O—C₁₋₄ halogen substituted alkyl, and NR^{b}R^{c}, or (2) C(O)-aryl optionally substituted with 1 to 3 groups independently selected from C₁₋₄ alkyl, halogen, nitro, cyano, OR^{d}, O—C₁₋₄ halogen substituted alkyl, and NR^{b}R^{c}.

In one embodiment of formula Id, R² is SO₂-aryl optionally substituted with 1 to 3 groups independently selected from C₁₋₄ alkyl, halogen, nitro, cyano, OR^{d}, O—C₁₋₄ halogen substituted alkyl, and NR^{b}R^{c}; and X is —C(O)NH—.

In a tenth subset of formula I are compounds of the formula Ie: 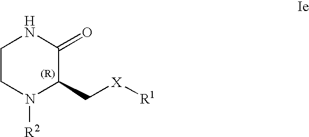
Wherein X, R¹ and R² are as defined under formula I.

When a variable occurs more than once, the definition for each occurrence is independent of the definition of the others; for example in —NR^{d}R^{d}, the R^{d} groups may be the same (e.g., —NH₂) or different (—NHCH₃). Unless otherwise stated, the following terms have the meanings indicated below:

“Alkyl” as well as other groups having the prefix “alk” such as, for example, alkoxy, alkanoyl, alkenyl, alkynyl and the like, means carbon chains which may be linear or branched or combinations thereof having the prescribed number of carbon atoms. Examples of alkyl groups include methyl, ethyl, propyl, isopropyl, butyl, sec- and tert-butyl, pentyl, hexyl, heptyl and the like.

“Aryl” means mono- or bicyclic aromatic rings containing only carbon atoms, or a benzene fused to a monocyclic cycloalkyl or monocyclic heterocyclyl group in which the point of attachment is on the benzene portion. Examples of aryl include phenyl, naphthyl, indanyl, indenyl, tetrahydronaphthyl, 5-, 6-, 7- or 8-tetrahydroquinolinyl, 5-, 6-, 7- or 8-quinolinyl, and the like.

“Cycloalkyl” means carbocycles having the prescribed number of ring carbon atoms and containing no heteroatoms. Examples of cycloalkyl include cyclopropyl, cyclobutyl, cyclopentyl and cyclohexyl.

“Halogen substituted alkyl” means an alkyl group substituted with at least one halogen atom and includes up to perhaloalkyl.

“Halogen” means fluorine, chlorine, bromine and iodine.

“Heteroaryl” means a mono- or bicyclic aromatic ring containing at least one heteroatom selected from N, O and S, with each ring containing 5 to 6 atoms, and where a bicyclic heteroaryl contains a benzene ring, the point of attachment is on the non-benzene portion.

“Heterocyclyl” means mono- or bicyclic aromatic, partially saturated, and saturated rings containing at least one heteroatom selected from N, S and O, each of said ring having from 3 to 10 atoms, and where a bicyclic heterocyclyl contains a benzene ring, the point of attachment is on the non-benzene portion. Examples of “heterocyclyl” include pyrrolidinyl, piperidinyl, piperazinyl, imidazolinyl, imidazolidinyl, 2,3-dihydrofuro(2,3-b)pyridyl, benzoxazinyl, tetrahydroquinolinyl, tetrahydroisoquinolinyl, dihydroindolyl, tetrahydronaphthyridinyl, pyridyl, pyrimidinyl, pyrrolyl, imidazolyl, triazolyl, thiazolyl, thiadiazolyl, isothiazolyl, oxazolyl, oxadiazolyl, isoxazolyl, naphthyridinyl, quinolinyl, isoquinolinyl, tetrazolyl, furanyl, triazinyl, thienyl, pyridazinyl, pyrazinyl, benzoxazolyl, benzothiazolyl, benzimidazolyl, benzofuranyl, benzothiophenyl, indolyl, and the like. The term “heterocyclyl” encompasses heteroaryl.

“Optionally substituted” is intended to include both substituted and unsubstituted. Thus, for example, optionally substituted aryl could represent a pentafluorophenyl or a phenyl ring.

Optical Isomers-Diastereomers-Geometric Isomers-Tautomers

Compounds described herein contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formula I is shown without a definitive stereochemistry at certain positions. The present invention includes all stereoisomers of Formula I and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I may be obtained by stereospecific synthesis using optically pure starting materials or reagents of known configuration.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

Compounds of formula I encompass those labeled with a radioisotope such as 35S. Radiolabeled compounds are utilized in biological assays as described herein.

Salts

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

Prodrugs

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with a compound of formula I or with a compound which converts to a compound of formula I in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

Pharmaceutical Compositions

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

The pharmaceutical compositions of the present invention comprise a compound represented by Formula I (or pharmaceutically acceptable salts thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The following are examples of representative pharmaceutical dosage forms for the compounds of Formula I:

| Inj. Suspension (I.M.) | mg/mL | Tablet | mg/tab. | Capsule | mg/cap. |
|---|---|---|---|---|---|
| Cmpd of Formula I | 10 | Cmpd of Formula I | 25 | Cmpd of Formula I | 25 |
| Methylcellulose | 5.0 | Microcryst. Cellulose | 415 | Lactose Powder | 573.5 |
| Tween 80 | 0.5 | Povidone | 14.0 | Magnesium Stearate | 1.5 |
| Benzyl alcohol | 9.0 | Pregelatinized Starch | 43.5 | | 600 |
| Benzalkonium chloride | 1.0 | Magnesium Stearate | 2.5 | | |
| Water for injection to a total | | | 500 | | |
| volume of 1 mL | | | | | |

Utilities

Compounds of this invention are antagonists or inverse agonists of bradykinin receptor, in particular the bradykinin B1 receptor, and as such are useful in the treatment and prevention of diseases and conditions mediated through the bradykinin receptor pathway such as pain and inflammation. The compounds would be effective in the treatment or prevention of pain including, for example, visceral pain (such as pancreatitis, interstitial cystitis, renal colic), neuropathic pain (such as postherpetic neuralgia, nerve injury, the “dynias”, e.g., vulvodynia, phantom limb pain, root avulsions, painful traumatic mononeuropathy, painful polyneuropathy), central pain syndromes (potentially caused by virtually any lesion at any level of the nervous system), and postsurgical pain syndromes (eg, postmastectomy syndrome, postthoracotomy syndrome, stump pain)), bone and joint pain (osteoarthritis), repetitive motion pain, dental pain, cancer pain, myofascial pain (muscular injury, fibromyalgia), perioperative pain (general surgery, gynecological), chronic pain, dysmennorhea, as well as pain associated with angina, and inflammatory pain of varied origins (e.g. osteoarthritis, rheumatoid arthritis, rheumatic disease, teno-synovitis and gout).

Further, the compounds of this invention can also be used to treat hyperreactive airways and to treat inflammatory events associated with airways disease e.g. asthma including allergic asthma (atopic or non-atopic) as well as exercise-induced bronchoconstriction, occupational asthma, viral- or bacterial exacerbation of asthma, other non-allergic asthmas and “wheezy-infant syndrome”. Compounds of the present invention may also be used to treat chronic obstructive pulmonary disease including emphysema, adult respiratory distress syndrome, bronchitis, pneumonia, allergic rhinitis (seasonal and perennial), and vasomotor rhinitis. They may also be effective against pneumoconiosis, including aluminosis, anthracosis, asbestosis, chalicosis, ptilosis, siderosis, silicosis, tabacosis and byssinosis.

Compounds of the present invention may also be used for the treatment of inflammatory bowel disease including Crohn's disease and ulcerative colitis, irritable bowel syndrome, pancreatitis, nephritis, cystitis (interstitial cystitis), uveitis, inflammatory skin disorders such as psoriasis and eczema, rheumatoid arthritis and edema resulting from trauma associated with burns, sprains or fracture, cerebral edema and angioedema. They may be used to treat diabetic vasculopathy, diabetic neuropathy, diabetic retinopathy, post capillary resistance or diabetic symptoms associated with insulitis (e.g. hyperglycemia, diuresis, proteinuria and increased nitrite and kallikrein urinary excretion). They may be used as smooth muscle relaxants for the treatment of spasm of the gastrointestinal tract or uterus. Additionally, they may be effective against liver disease, multiple sclerosis, cardiovascular disease, e.g. atherosclerosis, congestive heart failure, myocardial infarct; neurodegenerative diseases, eg. Parkinson's and Alzheimers disease, epilepsy, septic shock e.g. as anti-hypovolemic and/or anti-hypotensive agents, headache including cluster headache, migraine including prophylactic and acute use, closed head trauma, cancer, sepsis, gingivitis, osteoporosis, benign prostatic hyperplasia and hyperactive bladder. Animal models of these diseases and conditions are generally well known in the art, and may be suitable for evaluating compounds of the present invention for their potential utilities.

Compounds of the present invention are also useful as research tools (in vivo, in vitro and ex vivo). In one aspect a compound of the present invention is labeled with a radionuclide, preferably ³⁵S, and used in a brain receptor occupancy assay to assess the ability of test compounds to penetrate the blood brain barrier as well as the ability to distribute into the tissue and bind to the receptor. One such receptor occupancy assay using transgenic animal expressing human bradykinin B1 receptor is described in PCT Published Application WO03/016495 (Hess, et al), which is hereby incorporated by reference in its entirety.

Dosage and Administration

The compounds of this invention are useful in the treatment of pain and inflammation by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

The compounds would be effective in the treatment or prevention of pain including, for example, bone and joint pain (osteoarthritis), repetitive motion pain, dental pain, cancer pain, myofascial pain (muscular injury, fibromyalgia), perioperative pain (general surgery, gynecological) and chronic pain by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

In particular, inflammatory pain such as, for example, inflammatory airways disease (chronic obstructive pulmonary disease) would be effectively treated by the compounds of this invention by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Further, the compounds of this invention can additionally be used to treat asthma, inflammatory bowel disease, rhinitis, pancreatitis, cystitis (interstitial cystitis), uveitis, inflammatory skin disorders, rheumatoid arthritis and edema resulting from trauma associated with burns, sprains or fracture by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used subsequent to surgical intervention (e.g. as post-operative analgesics) and to treat inflammatory pain of varied origins (e.g. osteoarthritis, rheumatoid arthritis, rheumatic disease, teno-synovitis and gout) as well as for the treatment of pain associated with angina, menstruation or cancer by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat diabetic vasculopathy, post capillary resistance or diabetic symptoms associated with insulitis (e.g. hyperglycemia, diuresis, proteinuria and increased nitrite and kallikrein urinary excretion) by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat inflammatory skin disorders such as psoriasis and eczema by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used as smooth muscle relaxants for the treatment of spasm of the gastrointestinal tract or uterus or in the therapy of Crohn's disease, ulcerative colitis or pancreatitis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Such compounds may be used therapeutically to treat hyperreactive airways and to treat inflammatory events associated with airways disease e.g. asthma, and to control, restrict or reverse airways hyperreactivity in asthma by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat intrinsic and extrinsic asthma including allergic asthma (atopic or non-atopic) as well as exercise-induced bronchoconstriction, occupational asthma, viral or bacterial exacerbated asthma, other non-allergic asthmas and “wheezy-infant syndrome” by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may also be effective against pneumoconiosis, including aluminosis, anthracosis, asbestosis, chalicosis, ptilosis, siderosis, silicosis, tabacosis and byssinosis was well as adult respiratory distress syndrome, chronic obstructive pulmonary or airways disease, bronchitis, allergic rhinitis, and vasomotor rhinitis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Additionally, they may be effective against liver disease, multiple sclerosis, atherosclerosis, Alzheimer's disease, septic shock e.g. as anti-hypovolemic and/or anti-hypotensive agents, cerebral edema, headache including cluster headache, migraine including prophylactic and acute use, closed head trauma, irritable bowel syndrome and nephritis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (1) morphine and other opiate receptor agonists including propoxyphene (Darvon); (2) non-steroidal antiinflammatory drugs (NSAIDs) including COX-2 inhibitors such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamnic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone), and the coxibs (celecoxib, valecoxib, rofecoxib and etoricoxib); (3) corticosteroids such as betamethasone, budesonide, cortisone, dexamethasone, hydrocortisone, methylprednisolone, prednisolone, prednisone and triamcinolone; (4) histamine H1 receptor antagonists such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, desloratadine, fexofenadine and levocetirizine; (5) histamine H2 receptor antagonists such as cimetidine, famotidine and ranitidine; (6) proton pump inhibitors such as omeprazole, pantoprazole and esomeprazole; (7) leukotriene antagonists and 5-lipoxygenase inhibitors such as zafirlukast, montelukast, pranlukast and zileuton; (8) drugs used for angina, myocardial ischemia including nitrates such as nitroglycerin and isosorbide nitrates, beta blockers such as atenolol, metoprolol, propranolol, acebutolol, betaxolol, bisoprolol, carteolol, labetalol, nadolol, oxprenolol, penbutolol, pindolol, sotalol and timolol, and calcium channel blockers such as diltiazam, verapamil, nifedipine, bepridil, felodipine, flunarizine, isradipine, nicardipine and nimodipine; (9) incontinence medications such as antimuscarinics, e.g., tolterodine and oxybutinin); (10) gastrointestinal antispasmodics (such as atropine, scopolamine, dicyclomine, antimuscarinics, as well as diphenoxylate); skeletal muscle relaxants (cyclobenzaprine, carisoprodol, chlorphenesin, chlorzoxazone, metaxalone, methocarbamol, baclofen, dantrolene, diazepam, or orphenadrine); (11) gout medications such as allopurinol, probenicid and coichicirie; (12) drugs for rheumatoid arthritis such as methotrexate, auranofin, aurothioglucose and gold sodium thiomalate; (13) drugs for osteoporosis such as alendronate and raloxifene; decongestants such as pseudoephedrine and phenylpropanolamine; (14) local anesthetics; (15) anti-herpes drugs such as acyclovir, valacyclovir and famcyclovir; and (15) anti-emetics such as ondansetron and granisetron.

Biological Evaluation

Assessing the Affinity of Selected Compounds to Bind to the Bradykinin B1 or B2 Receptor

Radioligand binding assays are performed using membranes from CHO cells that stably express the human, rabbit, rat, or dog B1 receptors or CHO cells that express the human B2 receptor. For all receptor types, cells are harvested from culture flasks in PBS/1 mM EDTA and centrifuged at 1000×g for 10 minutes. The cell pellets are homogenized with a polytron in ice cold 20 mM HEPES, 1 mM EDTA, pH 7.4 (lysis buffer) and centrifuged at 20,000×g for 20 minutes. The membrane pellets are rehomogenized in lysis buffer, centrifuged again at 20,000×g and the final pellets are resuspended at 5 mg protein/ml in assay buffer (120 mM NaCl, 5 mM KCl, 20 mM HEPES, pH 7.4) supplemented with 1% BSA and frozen at −80° C.

On the day of assay, membranes are centrifuged at 14,000×g for 5 minutes and resuspended to the desired protein concentration in assay buffer containing 100 nM enaliprilat, 140 μg/mL bacitracin and 0.1% BSA. 3H-des-arg10, leu9 kallidin is the radioligand used for the human and rabbit B1 receptors, 3H-des-arg10 kallidin is used for the rat and dog B1 receptors, and 3H-bradykinin is used to label the human B2 receptor.

For all assays, compounds are diluted from DMSO stock solutions with 4 μL added to assay tubes for a final DMSO concentration of 2%. This is followed by the addition of 100 μL radioligand and 100 μL of the membrane suspension. Nonspecific binding for the B1 receptor binding assays is determined using 1 μM des-arg10 kallidin and nonspecific binding for the B2 receptor is determined with 1 μM bradykinin. Tubes are incubated at room temperature (22° C.) for 60 minutes followed by filtration using a Tomtec 96-well harvesting system. Radioactivity retained by the filter is counted using a Wallac Beta-plate scintillation counter.

The compounds of this invention have affinity for the B1 receptor in the above assay as demonstrated by results of less than 5 μM. It is advantageous that the assay results be less than 1 μM, even more advantageous for the results be less than 0.5 μM. It is further advantageous that compounds of this invention have affinity for the bradykinin B1 receptor over the bradykinin B2 receptor; more advantageously, the affinity for the B1 receptor is at least 10 fold, and preferably over 100 fold, over that for the B2 receptor.

Assay for Bradykinin B1 Antagonists

B1 agonist-induced calcium mobilization was monitored using a Fluorescence Imaging Plate Reader (FLIPR). CHO cells expressing the B1 receptor were plated in 96 or 384 well plates and allowed to incubate in Iscove's modified DMEM overnight. Wells were washed two times with a physiological buffered salt solution and then incubated with 4 uM Fluo-3 for one hour at 37° C. The plates were then washed two times with buffered salt solution and 100 uL of buffer was added to each well. Plates were placed in the FLIPR unit and allowed to equilibrate for two minutes. The test compound was then added in 50 ul volumes followed five minutes later by 50 ul of agonist (des-arg10 kallidin). Relative fluorescence peak heights in the absence and presence of antagonist were used to calculate the degree of inhibition of the B1 receptor agonist response by the test compound. Eight to ten concentrations of test compound were typically evaluated to construct an inhibition curve and determine IC50 values using a four-parameter nonlinear regression curve fitting routine.

Assay for Bradykinin Inverse Agonists

Inverse agonist activity at the human B1 receptor was evaluated using transiently transfected HEK293 cells. One day following transfection cell flasks were labeled overnight with 6 uCi/ml [³H]myo-inositol. On the day of assay, the media was removed and the attached cells were gently rinsed with 2×20 ml of phosphate-buffered saline. Assay buffer (HEPES buffered physiological salts, pH 7.4) was added and the cells were detached by tapping of the flask. The cells were centrifuged at 800×g for five minutes and resuspended at 1×10⁶ cells/ml in assay buffer supplemented with 10 mM lithium chloride. After 10 minutes at room temperature, one-half ml aliquots were distributed to tubes containing test compound or vehicle. After an additional 10 minutes the tubes were transferred to a 37° C. water bath for 30 minutes. The incubation was terminated by the addition of a 12% perchloric acid solution and the tubes were placed on ice for 30 minutes. The acid was then neutralized with KOH and the tubes centrifuged to pellet precipitated material. [³H]Inositol monophosphate formed was recovered by standard ion exchange chromatographic techniques and quantitated by liquid scintillation counting. Inverse agonist activity was determined by the degree to which a test compound reduced basal (cells incubated with vehicle) levels of [³H]inositol monophosphate accumulation.

The following abbreviations are used in the application: Ac=acetyl; Bn=benzyl; BOC=t-butyloxycarbonyl; BOP=(benzotriazol-1-yloxy)tris(dimethylamino)phosphonium hexafluorophosphate; EDCI=N-ethyl-N′-(3-dimethylaminopropyl)carbodiimide HCl; ES=electron spray; Et=ethyl; EtOAc=ethyl acetate; HOBT=1-hydroxybenzotriazole; LCMS=liquid chromatography mass spectrometry; Me=methyl; NMR=nuclear magnetic resonance; Ph=phenyl; THF=tetrahydrofuran.

Compounds of the present invention may be prepared according to the general procedures described in Schemes 1 to 7, and in the Examples. The procedures are provided as examples only; variations on the procedures described (for example, reagents used, sequence of reaction steps, protection/deprotection of functional groups, reaction conditions, solvents), as well as further elaboration (for example functional group manipulation, carbon chain lengthening) are within the skills of a synthetic organic chemist.

Scheme 1 shows the preparaion of 2-substituted-3-oxopiperazine derivatives (ii). A suitably di-protected 2-substituted-2-[(2-aminoethyl)amino]acetic acid (i) is first deprotected to provide a free amino group, and then treated with a base such as triethylamine to provide the ketopiperazine (ii). The protecting groups PG₁ and PG₂ may be any conventional amino and carboxyl protecting groups; for example t-butoxycarbonyl as amino protecting group, and alkyl or benzyl ester as carboxyl protection group. The substituent V may be an —X—R¹ group or it may be a precursor to —X—R¹ such as a carboxylate or a sulfanyl group, which can be further elaborated to provide compounds of formula I.

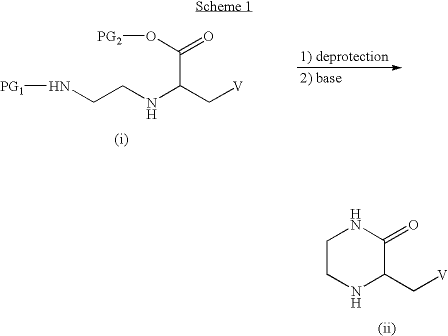

Scheme 2 provides examples of compounds of formula (i) and their preparation. Protected aminoacetaldehyde (iii) is reacted with an α-substituted-α-amino acid (iva or ivb) under reductive amination conditions, for example using sodium cyanoborohydride, to provide 2-substituted-2-[(2-aminoethyl)amino]acetic acids (ia) or (ib), respectively.

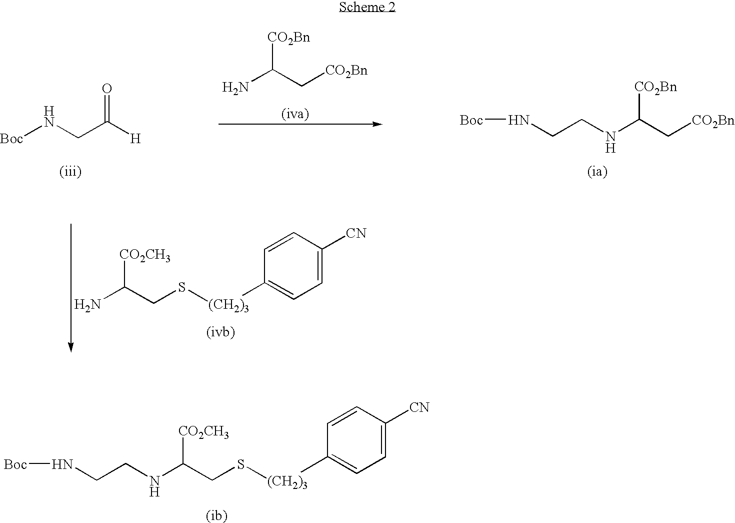

In Scheme 3,3-oxo-2-piperazineacetic acid derivative (v), where R is an ester group such as methyl, ethyl or benzyl, may be deprotected to provide the corresponding carboxylic acid (vi); or (v) may be reduced to provide the corresponding alcohol (vii). Deprotection of the carboxyl group may be accomplished by, for example, hydrolysis of alkyl ester or hydrogenolysis of benzyl ester, and the like. Reduction of compound (v) may be accomplished by using e.g. a lithium aluminum hydride reagent such as LiAlH(tBuO)₃ to give the corresponding alcohol (vi).

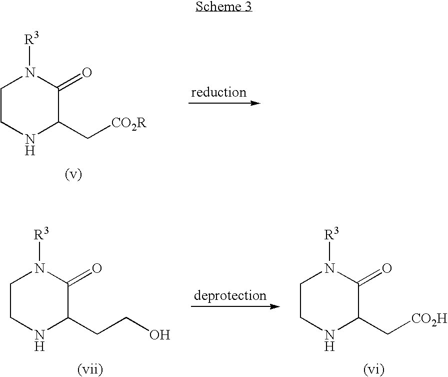

2-Hydroxyalkyl-3-oxopiperazines (viii) may be further elaborated to provide other intermediates useful in the preparation of compounds of formula I, as shown in Scheme 4. The hydroxy group is converted to the mesylate (ix) using mesyl chloride. Treatment of the mesylate with sodium azide provides the corresponding azido derivative (xi), which upon hydrogenation, gives the amine compound (xiii). Treatment of the mesylate (ix) with potassium thioacetate gives the thioate (x), which upon hydrolysis, yields the corresponding thiol (xii).

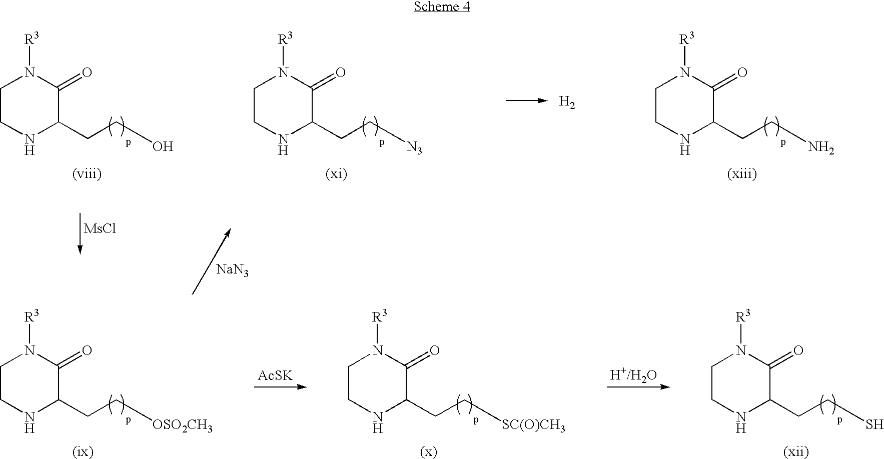

Scheme 5 illustrates various derivatization of functional groups of intermediates shown in Schemes 2 and 3 to provide compounds of formula I. The reactions such as amide and ester bond formation, nucleophilic substitution, oxidation, may be achieved using conventional synthetic methods well known in the art. Compound (xiv) may react with R²-L, where L is a suitable leaving group such as halide to provide (xv), which is then subject to derivatization at the 2-sidechain of the piperazine ring to provide (xva-xve); or alternatively, derivatization at the 2-sidechain is performed first to provide compounds (xiva-xive), which are then treated with R²-L to provide the corresponding (xva-xvb) compounds.

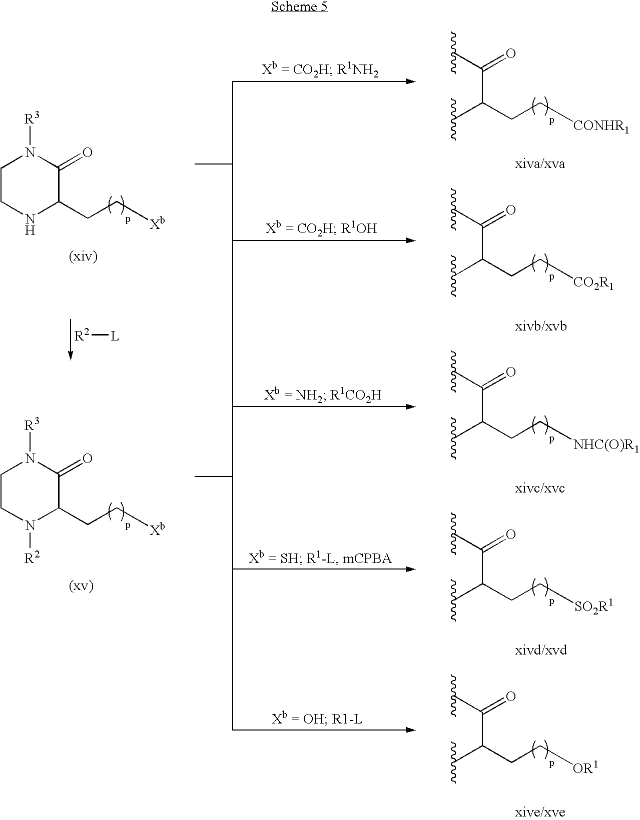

In Scheme 6, reacting 2-allyl-3-oxopiperazine (xvi) or (xvii) with an alkene in the presence of Grubb's catalyst gives the corresponding unsaturated compound (xvia) or (xviia), which upon hydrogenation, provides the saturated compound (xvib) or (xviib), respectively.

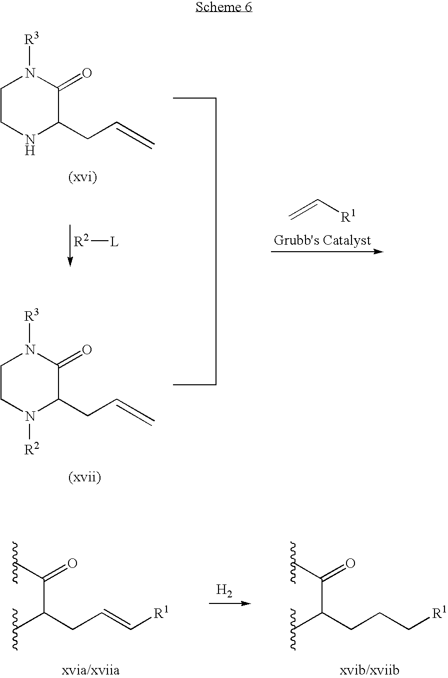

Benzonitriles may be converted to imidazolidinyl- and imidazolyl-substituted phenyl groups as illustrated in Scheme 7. The benzonitrile moiety may be linked to the ketopiperazine core, or it may be part of a precursor molecule to be attached to the ketopiperazine. Treatment of benzonitrile with HCl and ethanol followed by 1,2-ethylenediamne and ethanol provides the corresponding 4,5-dihydro-2-imidazolyl-phenyl compound. Treatment with HCl and ethanol followed by 2-aminoacetaldehyde dimethyl acetal converts the benzonitrile to the corresponding 2-imidazolyl-phenyl compound. The benzonitrile is converted to the corresponding 1,2,4-triazol-3-yl-phenyl compound upon treatment with HCl and ethanol, followed by formyl hydrazine (H₂N—NH—C(O)H).

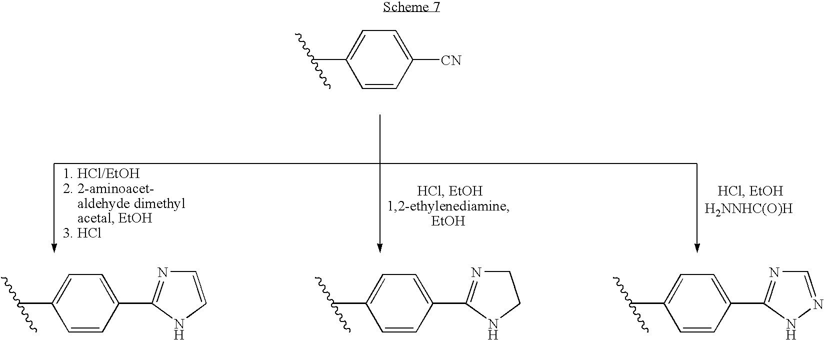

5-Oxo-2-piperazineacetic acid derivative may be prepared according to the procedure outlined in Scheme 8

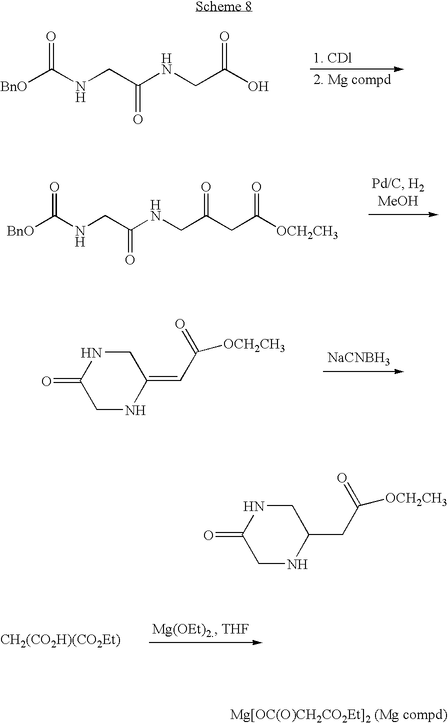

### REFERENCE EXAMPLES

### I. Preparation of 4-(2-aminoethyl)benzonitrile

To a solution of 4-(2-hydroxyethyl)benzonitrile (10.0 g, 67.94 mmol) in CH₂Cl₂ (175 mL) at 0° C. was added Et₃N (11.36 mL, 8.25 mmol) and MsCl (6.31 mL, 81.53 mmol). After stirring at 0° C. for 3.5 hours, the reaction was poured into water (100 ml) and separated. Organic layer was dried over Na₂SO₄, filtered and concentrated in vacuo. Crude residue was dissolved in DMF (100 mL) and NaN₃ (9.15 g, 140.72 mmol) and water (5 mL) were added. The resulting solution was heated to 125° C. After overnight stirring at 125° C., the crude reaction mixture was cooled, diluted with EtOAc (200 mL) and poured into water (150 mL). Organic layer was washed with water (5×150 mL), dried over sodium sulfate, filtered, and concentrated to give 4-(2-azidoethyl)benzonitrile which was used without purification.

A solution of 4-(2-azidoethyl)benzonitrile (500 mg, 2.90 mmol) in 1:1 EtOH/EtOAc (3 mL) at 0° C. was purged with N₂. Then Pd/C (440 mg) was added and a H₂ balloon was placed over the reaction. After stirring at 0° C. for two hours, the reaction mixture was filtered through a pad of celite and concentrated to give 4-2-aminoethyl)benzonitrile, which was used without further purification. LCMS (ES) 147.3 m/z (M+H)⁺.

### II. Preparation of 2-[4-(4,5-dihydro-1H-imidazol-2-yl)phenyl]ethanamine

A solution of 4-2-azidoethyl)benzonitrile (10.65 g, 172.19 mmol) in EtOAc (250 mL) at 0° C. was bubbled with HCl_{(g)} until saturation. Reaction was sealed and allowed to warm to room temperature slowly. After overnight stirring, the crude reaction mixture was concentrated under reduced pressure. The residue (10.1 g, 46.28 mmol) was taken up in EtOH and ethylene diamine (6.19 mL, 92.55 mmol) was added. After overnight stirring, the crude reaction mixture was concentrated, diluted with EtOAc (200 mL) and washed with water (3×150 mL). Organic layer was dried over sodium sulfate, filtered, and concentrated. Crude product was purified by flash chromatography on silica gel eluting with 10%-20% MeOH in CHCl₃ with 1% NH₄OH to give 900 mg (9%) of 2-[4-(4,5-dihydro-1H-imidazol-2-yl)phenyl]ethanamine. LCMS (ES) 216.3 m/z (M+H)⁺.

### III. Preparation of 4-(3-iodopropyl)benzonitrile

To a solution of 4-(3-hydroxyprop-1-ynyl)benzonitrile (2.0 g, 12.7 mmol) in EtOAc (25 mL), Pd/C (500 mg, 0.47 mmol) was added. A hydrogen balloon was placed over the reaction, and the reaction stirred for approximately four hours. An additional 200 mg (0.19 mmol) of Pd/C was added and the balloon was placed back over the reaction. Upon completion, the reaction mixture was filtered through a pad of celite and concentrated to give 4-(3-hydroxypropyl)benzonitrile which was used without further purification.

To a solution of 4-(3-hydroxypropyl)benzonitrile (1.8 g, 11.2 mmol) in CH₂Cl₂ (50 mL) at 0° C., Et₃N (1.06 mL, 13.4 mmol) and then MsCl (0.95 mL, 12.5 mmol) were added. Upon completion, the reaction mixture was poured into 0.5M HCl and extracted with EtOAc. The crude mesylate was dissolved in acetone and treated with NaI (3.0 g, 20.0 mmol) in the dark at room temperature. When complete, the reaction was purified by flash chromatography on silica gel eluting with 15% EtOAc in Hexanes to afford the title compound.

### IV. Preparation of 2-[4-(1H-imidazol-2-yl)phenyl]ethanamine

A solution of 4-(2-azidoethyl)benzonitrile (2.0 g, 11.62 mmol) in EtOH (50 mL) at 0° C. was saturated with HCl_{(g)}. Reaction mixture was allowed to warm to room temperature and stirred overnight. Concentration of the crude reaction mixture gave ethyl 4-(2-azidoethyl)-benzenecarboximidoate which was used without further purification.

Ethyl-4-(2-azidoethyl)benzenecarboximidoate (3.17 g, 14.55 mmol) and 2-amino-acetaldehyde dimethyl acetal (1.90 mL, 17.46 mmol) were mixed together in EtOH (20 mL) at room temperature. After 2.5 hours, 7 mL of HOAc were added. After overnight stirring, the reaction was heated to 50° C. and stirred for 3.5 hours before 1 mL of water was added. After an additional three hours of stirring at 50° C., the crude reaction mixture was diluted with EtOAc (200 mL) and washed with 10% Na₂CO₃ (2×50 mL) and brine (1×50 mL). The crude residue was taken up in water (20 mL) and concentrated HCl was added (20 mL). Resulting solution stirred at room temperature for three hours and was then heated to 80° C. for an additional one hour. Reaction mixture was cooled and K₂CO₃ (solid) was added until bubbling was no longer apparent upon addition. Crude material was extracted with CH₂Cl₂ (4×50 mL), dried over sodium sulfate, filtered, and concentrated. Purification by flash chromatography on silica gel eluting with 1%-5% MeOH in CH₂Cl₂ gave 910 mg (29%) of 2-[4-(2-azidoethyl)phenyl]-1H-imidazole.

To a solution of 2-[4-(2-azidoethyl)phenyl]-1H-imidazole (120 mg, 0.56 mmol) in EtOH, Pd/C (6 mg, 0.06 mmol) was added. A hydrogen balloon was placed over the reaction and the reaction was complete after approximately five hours. Reaction mixture was filtered through a pad of celite and concentrated to give 2-[4-(1H-imidazol-2-yl)phenyl]ethanamine which was used without further purification. LCMS (ES) m/z 188.4 (M+H)⁺.

The following examples are provided to illustrate the invention and are not to be construed as limiting the scope of the invention in any manner.

### Example 1

### N-{2-[4-(4,5-dihydro-1H-imidazol-2-yl)phenyl]ethyl}-2-[(2R)-3-oxo-1-(1,2,3,4-tetrahydroquinolin-8-ylsulfonyl)piperazin-2-yl]acetamide

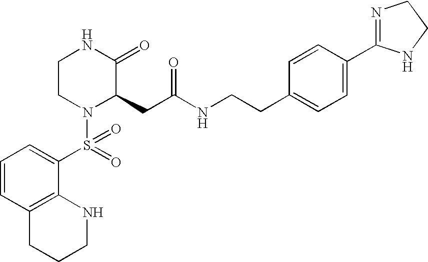

To a solution of methyl [(tert-butoxycarbonyl)amino]acetate, 1a (8.59 g, 45.45 mmol) in CH₂Cl₂ (150 mL) at −78° C., was added diisobutylaluminum hydride (100 mL, 1M solution in CH₂Cl₂). The reaction mixture stirred at −78° C. for approximately one hour before it was quenched with 1N HCl (104 mL). The reaction mixture was then warmed to 0° C., neutralized with saturated NaHCO₃, and further warmed to room temperature. After overnight stirring, the reaction mixture was filtered through celite, and partitioned bewtween CH₂Cl₂ and water. The combined organic phases were dried over sodium sulfate filtered, and concentrated. The crude product was distilled under high vacuum, collecting between 95° C. and 102° C., to give tert-butyl 2-oxoethylcarbamate.

L-aspartic acid dibenzyl ester p-toluenesulfonate (12.75 g, 26.26 mmol) was dissolved in CH₂Cl₂ and washed with aqueous 10% Na₂CO₃ and water. The organic phase was dried over sodium sulfate, filtered, and concentrated. The residue was taken up in 1,2-dichloroethane and tert-butyl 2-oxoethylcarbamate (4.18 g, 26.26) and sodium triacetoxyborohydride (8.35 g, 39.39 mmol) were added. Upon completion, the reaction mixture was quenched with saturated NaHCO₃ and extracted with CH₂Cl₂ (2×). The combined organic phases were dried over sodium sulfate, filtered, and concentrated. Silica gel chromatography (1:1 EtOAc/hexanes) gave dibenzyl (2R)-2-({2-[(tert-butoxycarbonyl)amino]-ethyl}amino)butanedioate, 1b.

HCl gas was bubbled into a solution of 1b (8.30 g, 18.18 mmol) in EtOAc at 0° C. for 15 minutes. Upon completion, N₂ gas was bubbled into the reaction mixture for approximately one hour before it was concentrated in vacuo. The resulting white solid was suspended in 1,2-dichloroethane (150 mL), Et₃N was added (8 mL, 57.40 mmol), and the resulting solution heated to 85° C. Additional Et₃N was added as necessary to further the progression of the reaction. Upon completion, the reaction mixture was diluted with CH₂Cl₂ (200 mL) and washed with water (1×), brine (1×), and saturated NaHCO₃ (1×). The organic phase was dried over sodium sulfate, filtered, and concentrated. Silica gel chromatography (3% MeOH: 30% CH₂Cl₂ saturated with NH₃: 67% CH₂Cl₂) gave benzyl [(2R)-3-oxopiperazin-2-yl]acetate, 2. LCMS (ES) m/z 249.3 (M+H)⁺.

To a solution of 2 (231 mg, 0.931 mmol) in pyridine (5 m L), 8-quinolinesulfonyl chloride (233 mg, 1.02 mmol) was added. After overnight stirring, the reaction mixture was concentrated to remove the pyridine and then partitioned between EtOAc and water. Combined organics were washed with brine, dried over sodium sulfate, filtered, and concentrated. The crude residue was taken up in EtOAc and Pd/C (10% by weight, 100 mg) was added. A hydrogen balloon was placed over the reaction mixture. After three hours, EtOH (1 mL) was added to the reaction mixture. The reaction mixture was quenched by filtering through celite, washed with EtOH and EtOAc, and concentrated. The crude material was re-subjected to the same reaction conditions using a 1:1 mixture of EtOH and EtOAc. After approximately two hours the reaction mixture was filtered through celite and concentrated to give [(2R)-3-oxo-1-(1,2,3,4-tetrahydroquinolin-8-ylsulfonyl)piperazin-2-yl]acetic acid, 3.

To a solution of 3 (136 mg, 0.386 mmol), 4-(2-aminoethyl)benzonitrile (62 mg, 0.424 mmol), and BOP (342 mg, 0.772 mmol), was added Et₃N. After overnight stirring, the reaction mixture was partitioned between EtOAc and water. The combined organic phases were washed with brine (1×), dried over sodium sulfate, filtered, and concentrated. Silica gel chromatography eluting with 3% MeOH in CH₂Cl₂ gave N-[2-(4-cyanophenyl)ethyl]-2-[(2R)-3-oxo-1-[1,2,3,4-tetrahydroquinolin-8-yl)sulfonyl]-piperazin-2-yl]acetamide, 4. LCMS (ES) m/z 482.3 (M+H)⁺.

HCl gas was bubbled into a solution of 4 (142 mg, 0.295 mmol) in EtOH at 0° C. for approximately 10 minutes. Reaction was capped and allowed to warm to room temperature. After overnight stirring, the reaction mixture was concentrated. Crude imidate was dissolved in EtOH (10 mL) and ethylene diamine (1.5 mL, 22.44 mmol) was added. Upon completion, the reaction mixture was concentrated and then partitioned between EtOAC and 10% Na₂CO₃. The combined organic phases were washed with water (1×), brine (1×), dried over sodium sulfate, filtered, and concentrated. Silica gel chromatography gave title compound, N-{2-[4-(4,5-dihydro-1H-imidazol-2-yl)phenyl]ethyl}-2-[(2R)-3-oxo-1-(1,2,3,4-tetrahydroquinolin-8-ylsulfonyl)piperazin-2-yl]acetamide, 5. ¹HNMR (400 MHz, CD₃OD) δ 7.70 (d, J=8.04 Hz, 2H), 7.46 (d, J=7.59 Hz, 1H), 7.32 (d, J=8.04 Hz, 2H), 7.09 (d, J=6.68 Hz, 1H), 6.54 (t, J=7.72 Hz, 1H), 4.76 (t, J=5.71 Hz, 1H), 3.75 (s, 4H), 3.69 (d, J=14.17 Hz, 1H), 3.28-3.47 (m, 5H), 2.99-3.03 (m, 2H), 2.84 (t, J=7.13 Hz, 2H), 2.65-2.74 (m, 4H), 1.83 (t, J=6.03 Hz, 2H). LCMS (ES) m/z 525.45 M+)⁺.

### Example 2

### (3S)-3-[({3-[4-(4,5-dihydro-1H-imidazol-2-yl)phenyl]propyl}sulfonyl)methyl]-4-(2-naphthylsulfonyl)-piperazin-2-one

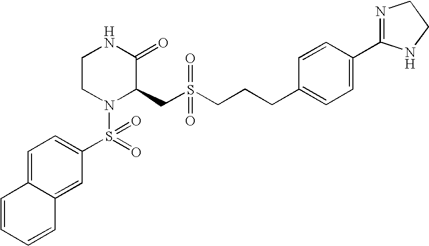

To a solution of methyl (2R)-2-[(tert-butoxycarbonyl)amino]-3-hydroxypropanoate, 6 (5.00 g, 22.81 mmol) in THF (110 mL) at 0° C., Et₃N (6.36 mL, 45.61 mmol) and methanesulfonyl chloride (3.53 mL, 45.61 mmol) were added. After stirring for 2 hours at 0° C., the solvent was removed under reduced pressure. The crude residue was diluted with CH₂Cl₂ and washed with a half-brine solution and then with brine. The organic phase was dried over sodium sulfate, filtered, and concentrated. Silica gel chromatography (1-4% MeOH/CH₂Cl₂) gave methyl (2R)-2-[(tert-butoxy-carbonyl)amino]-3-[(methylsulfonyl)oxy]propanoate.

To a solution of methyl (2R)-2-[(tert-butoxycarbonyl)amino]-3-[(methylsulfonyl)-oxy]propanoate (4.78 g, 16.08 mmol) in acetone (160 mL) was added potassium thioacetate (9.18 g, 80.41 mmol). The resulting mixture was heated to reflux for 2 hours and then cooled to ambient temperature. The precipitates in the mixture were filtered off and the filtrate was concentrated to an oily residue. Silica gel chromatography of the crude oil (0.5-2% MeOH in CH₂Cl₂) gave methyl (2S)-3-(acetylthio)-2-[(tert-butoxycarbonyl)amino]propanoate, 7.

Methanol (67 mL) was added to a flask containing 7 (1.00 g, 3.61 mmol), 4-(3-iodopropyl)benzonitrile (0.98 g, 3.61 mmol), and potassium carbonate (0.50 g, 3.61 mmol). After overnight stirring, the reaction mixture was concentrated, and the crude residue was partitioned between CH₂Cl₂ and water. The pH of the aqueous layer was adjusted to ˜5 with 1N HCl. The organic phase was dried over sodium sulfate, filtered, and concentrated. The crude product was purified via silica gel chromatography eluting with 1% MeOH in CH₂Cl₂ to give methyl (2S)-2-[(tert-butoxycarbonyl)amino]-3-{[3-(4-cyanophenyl)propyl]thio}propanoate, 8.

To a solution of methyl (2S)-2-[(tert-butoxycarbonyl)amino]-3-{[3-(4-cyanophenyl)-propyl]thio}propanoate (1.27 g, 3.35 mmol) in EtOAc (35 mL) at 0° C. was bubbled HCl gas for approximately five minutes. The reaction mixture was then sealed and stirred at 0° C. for thirty minutes. Concentration of the reaction mixture gave methyl (2S)-2-amino-3-{[3-(4-cyano-phenyl)propyl]thio}propanoate hydrochloride.

The free-base of methyl (2S)-2-amino-3-{[3-(4-cyanophenyl)propyl]thio}propanoate hydrochloride (0.60 g, 2.15 mmol) and tert-butyl 2-oxoethylcarbamate (0.34 g, 2.15 mmol) were dissolved in MeOH (20 mL). To this stirred solution was added sodium cyanoborohydride (1M in THF, 2.37 ml, 2.37 mmol). After overnight stirring, the reaction mixture was concentrated. The residue was diluted with CH₂Cl₂ and filtered to remove solids. The concentrated filtrate was subjected to silica gel chromatography (1-10% MeOH/CH₂Cl₂) to give methyl (2S)-2-({2-[(tert-butoxycarbonyl)amino]ethyl}-amino)-3-{[3-(4-cyanophenyl)propyl]thio}propanoate, 9.

To a solution of 9 (400 mg, 0.949 mmol) in EtOAc (10 mL) at 0° C. was bubbled HCl (g) for approximately five minutes. The reaction mixture was then sealed and stirred at 0° C. for thirty minutes. Concentration of the reaction mixture gave methyl (2S)-2-[(2-aminoethyl)amino]-3-{[3-(4-cyanophenyl)propyl]thio}propanoate hydrochloride.

Methyl (2S)-2-[(2-aminoethyl)amino]-3-{[3-(4-cyanophenyl)propyl]thio}propanoate hydrochloride (0.34 g, 0.95 mmol) and Et₃N (0.29 g, 2.85 mmol) were dissolved in CH₂Cl₂ (10 mL). The resulting solution stirred at room temperature for 2 hours, was then heated to reflux for 3 hours, and finally cooled to ambient temperature for overnight stirring. The reaction mixture was concentrated and the residue subjected to silica gel chromatography (1-10% MeOH(CH₂CH₂) to give 4-[3-({[(2S)-3-oxopiperazin-2-yl]methyl}thio)propyl]benzonitrile, 10.

To a solution of 10 (90 mg, 0.311 mmol) in CH₂Cl₂ (4 mL), 2-naphthalenesulfonyl chloride (106 mg, 0.47 mmol) and Et₃N (0.07 mL, 0.47 mmol) were added. After overnight stirring, the reaction mixture was concentrated and the crude residue purified by silica gel chromatography (1%-4% MeOH/CH₂Cl₂). Re-purification using the same conditions gave 4-[3-({[(2S)-1-(2-naphthylsulfonyl)-3-oxopiperazin-2-yl]methyl}thio)propyl]benzonitrile, 11.

To a solution of 11 (103 mg, 0.22 mmol) in EtOH (3 mL) at 0° C. was bubbled HCl (g) for approximately 4 minutes. The reaction flask was sealed and the reaction mixture stirred at 0° C. for approximately 30 minutes before being warmed to room temperature. After overnight stirring, the reaction mixture was concentrated. The resulting residue was taken up in EtOH (3 mL), cooled to 0° C., and ethylenediamine (0.03 mL, 0.43 mmol) was added. After stirring at 0° C. for ten minutes, the reaction mixture was warmed to room temperature for approximately one hour. The reaction mixture was then concentrated and purified using silica gel chromatography (1%-15% MeOH/CH₂Cl₂ with 10% ammonium hydroxide) to give (3S)-3-[({3-[4-(4,5-dihydro-H-imidazol-2-yl)phenyl]propyl}thio)-methyl]-4-(2-naphthylsulfonyl)piperazin-2-one.

(3S)-3-[({3-[4-(4,5-dihydro-1H-imidazol-2-yl)phenyl]propyl}thio)methyl]-4-2-naphthylsulfonyl)piperazin-2-one (80 mg, 0.15 mmol) dissolved in CH₂Cl₂ (2 mL) was cooled to 0° C. Into this solution was added 3-chloroperoxybenzoic acid (79 mg, 0.46 mmol). After 30 min of stirring, the solvent was removed and the residue purified by reverse phase chromatography (10-60% CH₃CN/H₂O) to provide the title compound as a TFA salt. ¹HNMR (400 MHz, CD₃OD) δ 8.57 (s,1H), 8.10 (t, J=8.4 Hz, 2H), 8.01 (d, J=7.2 Hz, 1H), 7.91 (bd, J=8.4 Hz, 1H), 7.81 (d, J=8.4 Hz, 2H), 7.70 (m, 2H), 7.53 (d, J=8 Hz, 2H), 4.92-4.83 (m, 1H), 4.09 (s, 4H), 3.93-3.89 (m, 2H), 3.69-3.62 (m, 2H), 3.32-3.25 (m, 2H), 3.06 (m, 1H), 2.96-2.89 (m, 3H), 2.17 (m, 2H). LCMS (ES) m/z 555.4 (M+H)⁺.

### Example 3

### 2-{1-[(3-chlorophenyl)sulfonyl]-5-oxopiperazin-2-yl}-N-{2-[4-(4,5-dihydro-1H-imidazol-2-yl)phenyl]ethyl}acetamide

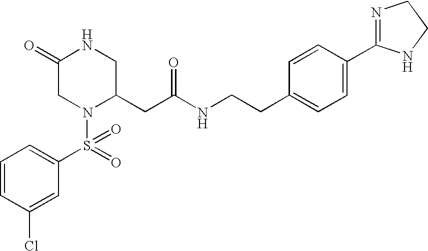

To a solution of (2-benzyloxycarbonylamino-acetylamino)-acetic acid, 13 (17.5 g, 65.73 mmol) in THF (164 mL), was added 1,1′-carbonyldiimidazole (11.4 g, 70.33 mmol). While this solution was stirring, the magnesium reagent, 13a was prepared by dissolving mono ethyl malonate (19.1 g, 144.6 mmol) in THF (180 mL) and adding magnesium ethoxide (8.27 g, 72.3 mmol). The resulting suspension stirred at room temperature for approximately 2.5 hours. The solvent was removed in vacuo and the residue azeotroped with toluene (1×50 mL) and THF (3×50 mL). The resulting magnesium salt was then taken up in THF (150 mL) and added to the imidazolide intermediate at room temperature. Upon completion, the reaction mixture was concentrated, re-dissolved in EtOAc (300 mL) and washed with 1M HCl (1×70 mL), 10% NaHCO₃ (1×70 mL), and brine (1×50 mL). The organic phase was dried over sodium sulfate, filtered, and concentrated to give ethyl 4-[({[(benzyloxy)-carbonyl]amino}acetyl)amino]-3-oxobutanoate, 14, which was used without further purification. LCMS (ES) m/z 337.2 (M+H)⁺.

To a solution of 14 (10.2 g, 30.36 mmol) in MeOH (100 mL), was added a suspension of Pd/C (1.1 g, 10% by weight) in MeOH (50 mL). The resulting suspension was placed on the Par Hydrogenator (53 psi) for approximately three days. The reaction mixture was filtered through celite, washing with MeOH. Concentration of the filtrate gave ethyl (2E)-(5-oxopiperazin-2-ylidene)ethanoate, 15, which was used without further purification. LCMS (ES) m/z 185.3 (M+H)⁺.

To a suspension of 15 (2.93 g, 15.9 mmol) in MeOH, acetic acid (1.28 ml, 22.4 mmol) was added. The resulting mixture was cooled to 0° C., NaCNBH₃ (31.8 mL, 1M in THF) was added via syringe, and the reaction mixture was warmed to room temperature. Upon completion, the reaction mixture was concentrated and azeotroped with toluene (1×25 mL). The residue was taken up in EtOAc (200 mL), and washed with 10% Na₂CO₃ (1×50 mL) and brine (1×20 mL). The organic phase was dried over sodium sulfate, filtered, and concentrated. Crude product was purified by silica gel chromatography (5%-15% MeOH/CHCl₃) to give ethyl (5-oxopiperazin-2-yl)acetate.

Ethyl (5-oxopiperazin-2-yl)acetate (325 mg, 1.75 mmol) was dissolved in dioxane (3.5 mL) and 1M Na₂CO₃ (1.75 mL) was added. The resulting solution was cooled to 0° C. and 3-chlorobenzenesulfonyl chloride (38 mg, 0.18 mmol) was added. The reaction mixture was allowed to warm to room temperature. Additional sulfonyl chloride was added (387 mg, 1.83 mmol) after approximately two hours. Upon completion, the reaction mixture was diluted with EtOAc (150 ml) and washed with brine (1×20 mL). The organic phase was dried over sodium sulfate, filtered, and concentrated to give ethyl {1-[(3-chlorophenyl)sulfonyl]-5-oxopiperazin-2-yl}acetate, 16, which was used without further purification. LCMS (ES) m/z 361.1 (M+H)⁺.

Compound 16 (262 mg, 0.728 mmol), 1N NaOH (0.764 mL), MeOH (2 mL), and THF (2 mL) were mixed together at 0° C. After approximately 15 minutes, the reaction mixture was warmed to room temperature. An additional equivalent of 1N NaOH (0.764 mL) was added after an hour. After completion, the pH of the reaction mixture was brought to a pH of 7.5 with 3M HCl. The solvents were removed in vacuo and the crude residue was azeotroped with toluene/THF (1:1, 7×30 mL) to give {1-[(3-chlorophenylsulfonyl]-5-oxopiperazin-2-yl}acetic acid, which was used without further purification.

A solution of {1-[(3-chlorophenyl)sulfonyl]-5-oxopiperazin-2-yl}acetic acid (241 mg, 0.728 mmol) and 2-[4-(4,5-dihydro-1H-imidazol-2-yl)phenyl]ethanamine (121 mg, 0.639 mmol) in DMF (3 mL) was cooled to 0° C. Then HOBt (98 mg, 0.728 mmol) and EDCI (250 mg, 0.728 mmol) were added. After approximately five minutes, NaHCO₃ (267 mg, 4.37 mmol) was added. After approximately fifteen minutes, the reaction mixture was warmed to room temperature until complete. The reaction mixture was partitioned between water (5 mL) and EtOAc (100 mL). After extracting an additional one time with EtOAc (50 mL), the organic phases were combined, dried over sodium sulfate, filtered, and concentrated. Silica gel chromatography eluting with 12% MeOH/CH₂Cl₂ to 15% MeOH/CH₂Cl₂ with 1.5% NH₄OH gave 2-{1-[(3-chlorophenyl)sulfonyl]-5-oxopiperazin-2-yl}-N-{2-[4-(4,5-hydro-1H-imidazol-2-yl)phenyl]ethyl}acetamide, 17. LCMS (ES) m/z 504.3 (M+H)⁺. ¹HNMR (400 MHz, DMSO) δ 7.86 (d, J=1.56 Hz, 1H), 7.76-7.81 (m, 2H), 7.75 (d, J=6.76 Hz, 2H), 7.64 (t, J=7.90 Hz, 1H), 7.26 (d, J=7.59 Hz, 2H), 4.38 (s, 1H), 3.99 (d, J=17.92 Hz, 1H), 3.72 (d, J=18.01 Hz, 1H), 3.68 (s, 4H), 3.24 (dd, J=12.94, 6.13 Hz, 2H), 3.16 (dd, J=4.80, 13.12 Hz, 1H), 2.96 (d, J=11.25 Hz, 1H), 2.71 (t, J=7.13 Hz, 2H), 2.32-2.44 (m, 2H).

Compounds shown in the following Tables were prepared following the general procedure described in Example 1. For the 2R-compounds, the ketopiperazine ring is formed as described in Example 1, and for the racemic compounds (2R/S), commercially available ethyl 2-(3-oxopiperazin-2-yl)acetate was used.

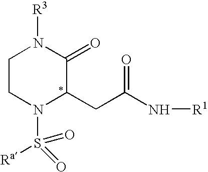

| | | | | | MS |
|---|---|---|---|---|---|
| Ex. | R³ | R¹ | R^{a′} | * | (ES) |
| 4 | H | (CH₂)₂-4-(4,5-dihydroimidazol-2-yl)-Ph | 2-naphthyl | R | 520.3 |
| 5 | H | (CH₂)₂-4-(4,5-dihydroimidazol-2-yl)-Ph | 3-Cl-Ph | R | 504.3 |
| 6 | H | (CH₂)₂-4-(2-imidazolyl)-Ph | 1,2,3,4-tetrahydro- | R | 523.3 |
| | | | quinolin-8-yl | | |
| 7 | H | (CH₂)₂-4-(2-imidazolyl)-Ph | 2-naphthyl | R | 518.3 |
| 8 | H | 4-[4-(1-piperidyl)-1-piperidyl]-Ph | 2-naphthyl | R/S | 590.5 |
| 9 | H | (CH₂)₂-4-(CH₂NH₂)-Ph | 1,2,3,4-tetrahydro- | R | 486.4 |
| | | | quinolin-8-yl | | |
| 10 | H | (CH₂)₄-2-(5,6,7,8-tetrahydro-1,8- | 2-naphthyl | R/S | 536.4 |
| | | naphthyridinyl) | | | |
| 11 | H | 4-(OCH₂CH₂NH₂)-Ph | 2-naphthyl | R/S | 482.5 |
| 12 | C(O)CF₃ | (CH₂)₂-4-(4,5-dihydroimidazol-2-yl)-Ph | 2-naphthyl | R/S | 602.4 |
| 13 | H | (CH₂)₂-4-(1,2,4-triazol-3-yl)-Ph | 2-naphthyl | R | 519.4 |
| 14 | H | (CH₂)₂-4-(4,5-dihydroimidazol-2-yl)-Ph | 2-naphthyl | R/S | 506.4 |
| 15 | H | (CH₂)₃-4-(2-amino)pyridyl | 2-naphthyl | R | 482.3 |
| 16 | CH₂Ph | (CH₂)₂-4-(4,5-dihydroimidazol-2-yl)-Ph | 2-naphthyl | R | 610.4 |
| 17 | C(O)-Ph | (CH₂)₂-4-(4,5-dihydroimidazol-2-yl)-Ph | CH₃ | R/S | 498.4 |
| 18 | SO₂CH₃ | (CH₂)₂-4-(4,5-dihydroimidazol-2-yl)-Ph | CH₃ | R/S | 472.3 |
| 19 | H | (CH₂)₂-4-(CN)-Ph | 1,2,3,4-tetrahydro- | R | 482.3 |
| | | | quinolin-8-yl | | |
| 20 | H | (CH₂)₅-O-3-pyridyl | 2-naphthyl | R/S | 511.4 |
| 21 | H | (CH₂)₂-4-(CH₂NHAc)-Ph | 1,2,3,4-tetrahydro- | R | 528.4 |
| | | | quinolin-8-yl | | |
| 22 | H | (CH₂)₂-4-(CN)-Ph | 2-naphthyl | R | 477.3 |
| 23 | H | (CH₂)₄-O-3-pyridyl | 2-naphthyl | R/S | 497.3 |
| 24 | CH₃ | (CH₂)₂-4-(4,5-dihydroimidazol-2-yl)-Ph | 2-naphthyl | R/S | 520.4 |
| 25 | C(O)-Ph | (CH₂)₂-4-(4,5-dihydroimidazol-2-yl)-Ph | 2-naphthyl | R/S | 610.5 |
| 26 | H | (CH₂)₃-O-3-pyridyl | 2-naphthyl | R/S | 483.3 |
| 27 | H | (CH₂)₆-O-3-pyridyl | 2-naphthyl | R/S | 525.4 |
| 28 | H | 4-((CH₂)₃NHCH₂CF₃)-Ph | 2-naphthyl | R/S | 563.2 |
| 29 | SO₂CH₃ | (CH₂)₂-4-(4,5-dihydroimidazol-2-yl)-Ph | 2-naphthyl | R/S | 584.5 |
| 30 | H | (CH₂)₄-O-2-pyridyl | 2-naphthyl | R/S | 497.3 |
| 31 | H | (CH₂)₅-O-2-pyridyl | 2-naphthyl | R/S | 511.4 |
| 32 | H | (CH₂)₂-morpholino | 2-naphthyl | R/S | 475.2 |
| 33 | H | (CH₂)₂-morpholino | 2-naphthyl | R/S | 461.2 |
| 34 | H | (CH₂)-4-(2-CO₂CH₃-Ph)-Ph | 2-naphthyl | R/S | 572.4 |
| 35 | H | (CH₂)₃-O-2-pyridyl | 2-naphthyl | R/S | 483.3 |
| 36 | H | 4-[4-(1-piperidyl)-1-piperidyl]-Ph | CH₃ | R/S | 478.4 |
| 37 | C(O)CH₃ | (CH₂)₂-4-(4,5-dihydroimidazol-2-yl)-Ph | CH₃ | R/S | 436.3 |

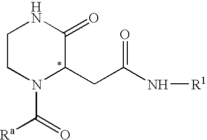

| | | | | MS |
|---|---|---|---|---|
| Ex. | R¹ | R^{a} | * | (ES) |
| 38 | (CH₂)₂-4-(4,5-dihydroimidazol-2-yl)-Ph | 3-Cl-Ph | R | 482.4 |
| 39 | (CH₂)-4-(2-CO₂CH₃-Ph)-Ph | CH₂CF₃ | R/S | 492.7 |
| 40 | (CH₂)-4-(2-CO₂CH₃-Ph)-Ph | CH₂CN | R/S | 449.7 |
| 41 | (CH₂)₂-4-(4,5-dihydroimidazol-2-yl)-Ph | 4-Cl-Ph | R/S | 468.3 |

1-15. (canceled)

16. A compound having the formula Ia and pharmaceutically acceptable salts thereof: 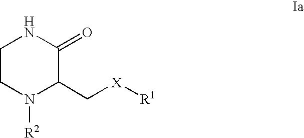
wherein
X is —C(O)NH— or SO₂;
R¹ is selected from (1) CH₂(CH₂)ₙO-aryl where aryl is optionally substituted with 1 to 3 groups independently selected from C₁₋₄ alkyl, halogen, nitro, cyano, OR^{d}, O—C₁₋₄ halogen substituted alkyl, and NR^{b}R_{c}, (2) CH₂(CH₂)ₙO-heteroaryl where heteroaryl is optionally substituted with 1 to 3 groups independently selected from C₁₋₄ alkyl, halogen, nitro, cyano, OR^{d}, O—C₁₋₄ halogen substituted alkyl, and NR^{b}R^{c}, (3) CH₂(CH₂)ₙNR^{b}R^{c}, (4) C₁₋₆ alkyl-Q wherein Q is (a) heterocyclyl optionally substituted with 1 to 3 groups independently selected from halogen, C₁₋₄ alkyl, C₁₋₄ halogen substituted alkyl, nitro, cyano, OR^{a}, and NR^{b}R^{c}; or (b) aryl optionally substituted with 1 to 3 groups independently selected from C₁₋₄ alkyl (optionally substituted with OR^{a}, NR^{b}R^{c}, NR^{b}C(O)R^{a}, or heterocyclyl), halogen, cyano, C(O)R^{a}, C(O)OR^{a}, OR^{a}, NR^{b}R^{c}, NR^{b}C(O)R^{a}, C(O)NR^{b}R^{c}, SR^{a}, S(O)ₘR^{a′}, aryl and heterocyclyl wherein said aryl and heterocyclyl are optionally substituted with 1 to 3 groups independently selected from C₁₋₄alkyl, C(O)OR^{a}, halogen, nitro, cyano, OR^{a}, and NR^{b}R^{c}, and (5) aryl optionally substituted with 1 to 3 groups independently selected from halogen, nitro, cyano, C(O)OR^{a}, OR^{a}, NR^{b}R^{c}, NR^{b}C(O)R^{a}, C(O)NR^{b}R^{c}, SR^{a}, S(O)ₘR^{a′}, (CH₂)ₙOR^{a}, (CH₂)ₙNR^{b}R^{c}, (CH₂)ₙNR^{b}C(O)OR^{a}, O(CH₂)ₙNR^{b}R^{c}, O(CH₂)ₙNR^{b}C(O)OR^{a}, C₁₋₄ alkyl, aryl and heterocyclyl wherein said aryl and heterocyclyl are optionally substituted with 1 to 3 groups independently selected from C₁₋₄alkyl, C(O)OR^{a}, halogen, nitro, cyano, OR^{a}, and NR^{b}R^{c}; and
R² is (1) SO₂-aryl optionally substituted with 1 to 3 groups independently selected from C₁₋₄ alkyl, halogen, nitro, cyano, OR^{d}, O—C₁₋₄ halogen substituted alkyl, and NR^{b}R^{c}, or (2) C(O)-aryl optionally substituted with 1 to 3 groups independently selected from C₁₋₄ alkyl, halogen, nitro, cyano, OR^{d}, O—C₁₋₄ halogen substituted alkyl, and NR^{b}R^{c};
R^{a} is selected from (1) hydrogen, (2) C₁₋₄ alkyl, (3) C₁₋₄ halogen substituted alkyl, (4) C₃₋₇ cycloalkyl, (5) aryl optionally substituted with 1 to 3 groups independently selected from C₁₋₄ alkyl, halogen, nitro, cyano, OR^{d}, O—C₁₋₄ halogen substituted alkyl, and NR^{b}R^{c}, and (6) heteroaryl optionally substituted with 1 to 3 groups independently selected from C₁₋₄ alkyl halogen, nitro, cyano, OR^{d}, O—C₁₋₄ halogen substituted alkyl, and NR^{b}R^{c};
R^{a′} is selected from (I) C₁₋₄ alkyl, (2) C₁₋₄ halogen substituted alkyl, (3) C₃₋₇ cycloalkyl, (4) aryl optionally substituted with 1 to 3 groups independently selected from C₁₋₄ alkyl, halogen, nitro, cyano, OR^{d}, O—C₁₋₄ halogen substituted alkyl, and NR^{b}R^{c}, and (5) heteroaryl optionally substituted with 1 to 3 groups independently selected from C₁₋₄ alkyl, halogen, nitro, cyano, OR^{d}, O—C₁₋₄ halogen substituted alkyl, and NR^{b}R^{c};
R^{b} and R^{c} are independently selected from (1) hydrogen, (2) C₁₋₄ alkyl, (3) C₁₋₄ halogen substituted alkyl, (4) C₃₋₇ cycloalkyl, and (5) aryl optionally substituted with 1 to 3 groups independently selected from C₁₋₄ alkyl, halogen, nitro, cyano, OR^{d}, O—C₁₋₄ halogen substituted alkyl, —C(O)OR^{d}, C(O)NR^{d}R^{d}, and NR^{d}R^{d}; or
R^{b}, R^{c} and the nitrogen atom to which they are attached together form a 4-7-membered ring optionally containing a heteroatom selected from O, S and N—R^{d},
R^{d} is hydrogen or C₁₋₄ alkyl:
n is an integer from 1 to 6; and
m is 1 or 2.

17. A compound of claim 16 wherein R² is SO₂-aryl optionally substituted with 1 to 3 groups independently selected from C₁₋₄ alkyl, halogen, nitro, cyano, OR^{d}, O—C₁₋₄ halogen substituted alkyl, and NR^{b}R^{c}.

18. A compound of claim 16 wherein R² is SO₂-(2-naphthyl) or SO₂-(1,2,3,4-tetrahydroquinolin-8-yl).

19. A compound of claim 16 wherein R¹ is selected from

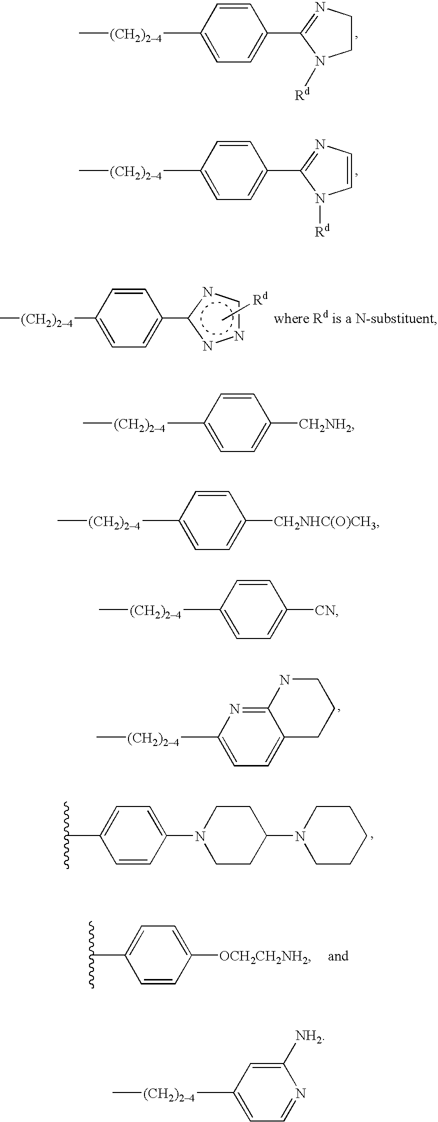

20. A compound of claim 19 wherein R² is SO₂-(2-naphthyl) or SO₂-(1,2,3,4-tetrahydroquinolin-8-yl).

21-31. (canceled)

32. A pharmaceutical composition comprising a therapeutically effective amount of a compuond of claim 16 and pharmaceutically acceptable excipients.

33. A method of treatment or prevention of pain and inflammation comprising administering to a patient in need of such treatment or prevention a therapeutically effective amount of a compound of claim 16.

34. (canceled)
